(19) United States
(12) Patent Application Publication
(10) Pub. No.: US 2007/0264655 A1
(43) Pub. Date: Nov. 15, 2007

(54) NUCLEIC ACID AUTOMATIC EXAMINING DEVICE

(75) Inventor: Hiroshi Netsu, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/739,772

(22) Filed: Apr. 25, 2007

(30) Foreign Application Priority Data
May 9, 2006 (JP) 2006-130297

(51) Int. Cl. C12Q 1/68; C12M 3/00

(52) U.S. Cl. 435/6; 435/287.2

(57) ABSTRACT

A nucleic acid automatic examining device implements a hybridization reaction step after implementing a nucleic acid amplification reaction step on a nucleic acid sample containing a target nucleic acid. The device includes a nucleic acid amplification unit, a hybridization reaction unit, a dispenser unit for moving the nucleic acid sample from the nucleic acid amplification unit to the hybridization reaction unit, and a detector for detecting presence or absence of a reaction cassette in the hybridization reaction unit. When the detector detects the absence of the reaction cassette, the device implements steps up to the nucleic acid amplification reaction step and the dispenser unit is stopped so that the device may not implement the subsequent hybridization reaction step.

### BACKGROUND OF THE INVENTION

1. Field of the Invention

The present invention relates to a nucleic acid automatic examining device for extracting a nucleic acid such as DNA contained in a sample such as blood to check whether the extracted nucleic acid contains something originating from disease-germs.

2. Related Background Art

As examples of a method of analyzing the base sequence of a nucleic acid and a method of quickly and accurately detecting a nucleic acid contained in a sample, there have been proposed a number of methods utilizing hybridization reaction using a probe immobilized carrier, such as a DNA microarray. While a sample may contain nucleic acids originating from various living bodies, the nucleic acid whose presence in the sample is to be ascertained will be referred to as a target nucleic acid. For example, in a case where infection with a specific disease-germ is to be checked, a blood sample is extracted blood and it is examined with respect to presence or absence of a nucleic acid having a base sequence characteristic to the specific disease-germs.

In a DNA microarray, a number of nucleic acids with base sequences complementary to those of target nucleic acids as probes are fixed at a high density to a substrate, such as a bead or a glass plate. A method of detecting a target nucleic acid by using such a DNA microarray includes a nucleic acid amplification step and a hybridization reaction step as described below.

In the nucleic acid amplification step, nucleic acids are amplified by a nucleic acid amplification method such as a PCR method. More specifically, nucleic acids contained in a sample are first extracted, and dissolved or dispersed in a predetermined solution. The solution is referred to as a nucleic acid sample. In the nucleic acid sample, various nucleic acids contained in the sample are dispersed or dissolved together with the target nucleic acid. When first and second primers are added to the nucleic acid sample and a temperature cycle is applied thereto, the first primer is bonded in a specific manner to a part of the target nucleic acid and the second primer is bonded in a specific manner to a part of a nucleic acid complementary to the target nucleic acid. When a double-stranded nucleic acid containing the target nucleic acid is bonded with the first and second primers, the target nucleic acid is copied through an elongation reaction. Since this reaction can be easily controlled by raising or lowering the temperature level, the total amount of the target nucleic acid can be sufficiently amplified by repeatedly effecting the reaction. When the amplification reaction is effected, other nucleic acids contained in the sample are simultaneously amplified together with the target nucleic acid.

After the nucleic acids in the nucleic acid sample have been sufficiently amplified, the substances other than the amplified nucleic acids, such as unreacted primers and nucleic acid fragments, are removed. According to known methods, the removal is effected by causing magnetic particles to adsorb the target nucleic acid or by using a column filter. A third primer is then added to the nucleic acid sample thus purified to contain only amplified nucleic acid, and a temperature cycle is applied thereto. The third primer is labeled with an enzyme, a fluorescent substance, a luminescent substance or the like, and binds in a specific manner to a part of the nucleic acid complementary to the target nucleic acid.

Here, the term “labeling” refers to a step of bonding an enzyme, a fluorescent substance, a luminescent substance or the like to the target nucleic acid. By labeling the target nucleic acid, it is possible to chemically, physically, or optically detect the target nucleic acid in the subsequent process steps. When the nucleic acid complementary to the target nucleic acid binds to the third primer, the target nucleic acid labeled with an enzyme, a fluorescent substance, a luminescent substance or the like is amplified through elongation reaction. When the target nucleic acid is contained in the nucleic acid sample, the labeled target nucleic acid is produced, and when the target nucleic acid is not contained in the nucleic acid sample, the labeled target nucleic acid is not produced.

The hybridization reaction step is a step in which the nucleic acid sample containing an enzyme, a fluorescent substance or the like which is amplified in the nucleic acid amplification step is spread on a DNA microarray, causing the nucleic acid sample to undergo hybridization reaction with the probes immobilized to the substrate. More specifically, the DNA microarray and the nucleic acid sample are brought into contact with each other, and then the temperature is raised to the hybridization temperature, which is maintained for a fixed period of time. When, at this time, the target nucleic acid complementary to the probe exists in the nucleic acid sample, the probe and the target nucleic acid undergo hybridization reaction to form a hybrid.

When the hybridization step is completed, the next step of detecting the target nucleic acid will begin. For example, when the target nucleic acid is labeled with a fluorescent substance, this fluorescent substance is excited by laser or the like to thereby generate fluorescence, whose intensity is measured. That is, by detecting the position and intensity of fluorescence on the DNA microarray, it is possible to ascertain the formation of a specific hybrid. By ascertaining the formation of the hybrid, it is possible to specify the nucleic acid contained in the nucleic acid sample.

Conventionally, there have been developed nucleic acid automatic examining devices in which the above-mentioned steps are respectively performed in units, conducting the examination in a single device. Japanese Patent Application Laid-Open No. 2003-274925 discloses a device in which a dispenser unit equipped with a plurality of chip nozzles is used to perform on a microplate the extraction of a nucleic acid and hybridization. Japanese Utility Model Application Laid-Open No. 03-76170 discloses a dispenser unit in which an operation of the dispenser unit for dispensing liquid into a container from a nozzle is controlled based on the information on presence/absence of a container obtained by a detector. With this construction, the nozzle is protected from damages, and the time loss due to futile operation of the dispenser unit is eliminated.

Usually, in the detection of a target nucleic acid utilizing hybridization reaction by using a DNA microarray, long time is required for the nucleic acid amplification step and the hybridization reaction step. In view of this, there has been developed a device in which the nucleic acid amplification step and the hybridization reaction step are totally automated.

When the nucleic acid sample is moved in each step or transferred to a next step, a dispenser unit, which is automatically controlled by a control section, is often used. However, when, in a nucleic acid automatic examining device, the presence/absence of a reaction cassette in a cassette holder is detected by a detector according to a prior-art technique and the dispenser unit is operated based on the detection signal from the detector, there may be involved futile standby time. That is, when the reaction cassette is not set at an accurate position and the dispenser unit stops, the movement of the nucleic acid sample in the nucleic acid amplification step is also stopped, resulting in generation of futile standby time.

### SUMMARY OF THE INVENTION

It is an object of the present invention to provide a nucleic acid automatic examining device for implementing a hybridization reaction step after implementing a nucleic acid amplification reaction step, in which a reduction in futile standby time is achieved.

The present invention provides a nucleic acid automatic examining device for implementing a hybridization reaction step after implementing a nucleic acid amplification reaction step on a nucleic acid sample containing a target nucleic acid, comprising: a nucleic acid amplification unit for implementing the nucleic acid amplification reaction step; a hybridization reaction unit having a cassette holder allowing installation of a reaction cassette for use in the hybridization reaction step performed on the nucleic acid sample; a movable dispenser unit having multiple nozzles capable of sucking and discharging the nucleic acid sample and moving the nucleic acid sample from the nucleic acid amplification unit to the hybridization reaction unit; a detector for detecting presence or absence of the reaction cassette in the cassette holder; and an alarm indicator for indicating an alarm, wherein the nucleic acid amplification unit and the hybridization reaction unit are operable in parallel and are controlled such that, when the detector detects the absence of the reaction cassette, the device implements steps up to the nucleic acid amplification reaction step and does not implement the subsequent hybridization reaction step while the alarm indicator indicates an alarm and the dispenser unit is stopped.

According to the present invention, it is possible to prevent contamination of an interior of the device attributable to a dispensing error due to a failure to set a predetermined number of reaction cassettes.

Further features of the present invention will become apparent from the following description of exemplary embodiments with reference to the attached drawings.

### BRIEF DESCRIPTION OF THE DRAWINGS

FIG. 1 is a perspective view illustrating a construction of a nucleic acid automatic examining device according to an embodiment of the present invention.

FIG. 2 is a perspective view of a cassette holder to which a reaction cassette according to the embodiment of the present invention is to be installed.

FIG. 3 is a schematic diagram in which a dispenser unit operating process and a nucleic acid sample examining process are shown side by side.

FIG. 4 is a 96-well plate which can be used in a nucleic acid automatic examining device according to an embodiment of the present invention.

FIG. 5 illustrates an example of a reaction cassette detection method which can be used in a nucleic acid automatic examining device according to an embodiment of the present invention.

FIG. 6 illustrates an example of a reaction cassette detection circuit which can be used in a nucleic acid automatic examining device according to the embodiment of the present invention.

FIG. 7 is a plan view showing the construction of a nucleic acid automatic examining device according to an embodiment of the present invention.

FIG. 8 is a block diagram showing a relationship between the units of a nucleic acid automatic examining device according to an embodiment of the present invention.

### DESCRIPTION OF THE EMBODIMENTS

Specific embodiments of the present invention will now be described in detail in accordance with the accompanying drawings.

In the following, a nucleic acid examining device according to the present invention will be described with reference to a case where a target nucleic acid is DNA. The target nucleic acid as an object of detection is not restricted to DNA; it may also be RNA.

The nucleic acid examining device of the present invention includes the following components (1) through (5):

(1) a nucleic acid amplification unit for effecting amplification reaction of a target nucleic acid;

(2) a hybridization reaction unit allowing installation of multiple reaction cassettes for effecting hybridization reaction of the target nucleic acid;

(3) a dispenser unit having multiple movable nozzles capable of sucking and discharging the target nucleic acid;

(4) a detector for detecting that the reaction cassettes are set at predetermined positions in the hybridization reaction unit; and

(5) a control section.

In the nucleic acid amplification unit, the target nucleic acid contained in the nucleic acid sample is amplified mainly by the PCR method. When the amount of target nucleic acid extracted is small, or when the amount of target nucleic acid extracted is sufficient but a large amount of substances other than the target nucleic acid are mixed therein and purification of the sample nucleic acid is necessary, it is possible to additionally arrange a DNA purification unit.

The hybridization reaction unit has an installation table on which there is provided a cassette holder allowing installation of multiple reaction cassettes. In this specification, the term “reaction cassette” represents a container having a liquid receiving port allowing dispensing of the nucleic acid sample from outside the container for the purpose of effecting hybridization reaction within the container.

The dispenser unit dispenses a nucleic acid solution containing the target nucleic acid to the reaction cassette. Further, when there are added units for performing the above-mentioned PCR amplification and the step of preparing a hybridization solution, it is also possible to add to the dispenser unit a construction allowing dispensing to a predetermined position of a reagent required in each step or a solution containing the target nucleic acid.

The reaction cassette is detachably installed in a cassette holder. It is possible to use multiple reaction cassettes as needed. The hybridization reaction unit is provided with the cassette holder so as to allow individual installation of multiple reaction cassettes. By using a detector, it is automatically determined whether the reaction cassettes installed in the cassette holder are installed at predetermined positions on the installation table of the hybridization reaction unit.

The necessary reagents for hybridization are respectively prepared in a well plate. While a well plate is naturally suitable for the reagent container, this should not be construed restrictively; it may be a container uniquely designed for the present device.

As the dispenser unit, one with multiple nozzles can be used. By using a dispenser unit having multiple dispensing nozzles, it is possible to dispense reagents and reaction liquids simultaneously to multiple reaction cassettes.

The detector is provided so as to be in contact with the cassette holder, and serves to detect whether the reaction cassettes are set at the predetermined positions in the hybridization reaction unit. It is also possible for this detector to be one capable of detecting whether all the reaction cassettes that can be set in the hybridization reaction unit are set at predetermined attachment positions.

Alternatively, it is also possible to detect whether a specific reaction cassette is set at a predetermined installation position. Further, it is possible for the detector to detect at one time whether a reaction cassette is set at a predetermined position, or to perform detection a number of times corresponding to the predetermined positions for the reaction cassettes, respectively; there are no particular limitations in this regard. The setting as to which reaction cassette, of the reaction cassettes set in the cassette holder, is to be detected, or whether all the reaction cassettes are to be detected, can be effected automatically according to a pre-set program.

In this device, it is possible to implement the amplification step and the hybridization reaction step in parallel. For example, when hybridization reaction is being implemented on one nucleic acid sample, it is possible to implement amplification on another nucleic acid sample.

All the operations described above are controlled by the control section.

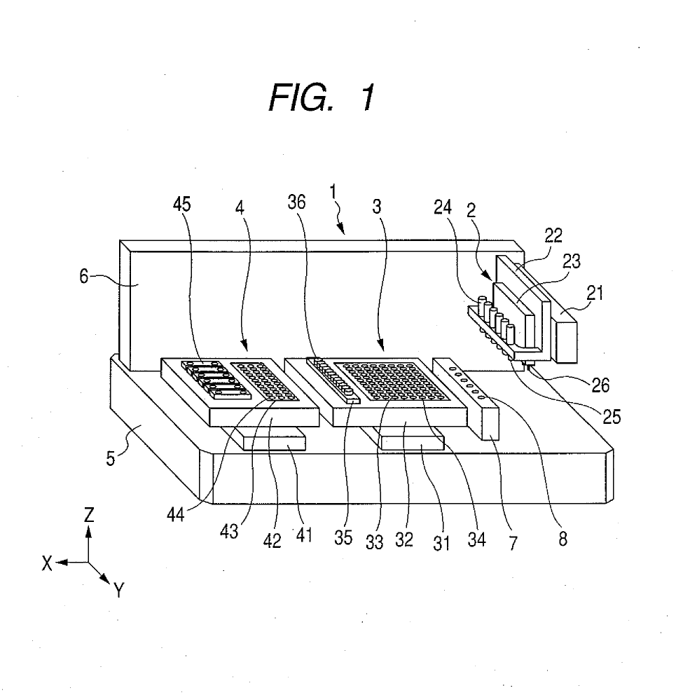

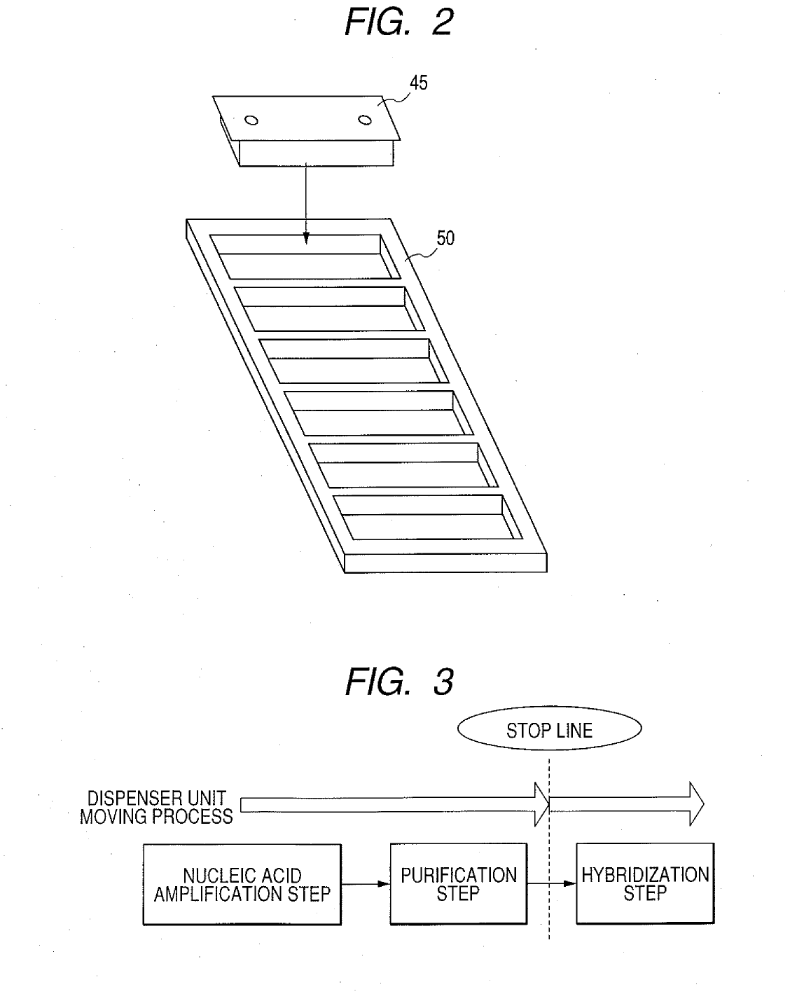

FIG. 1 is a perspective view illustrating the arrangement of each unit of a nucleic acid automatic examining device according to an embodiment of the present invention. First, a general construction of the nucleic acid automatic examining device will be described. A nucleic acid automatic examining device 1 includes a base stage 5, on which there are arranged a dispenser unit 2, a nucleic acid amplification unit 3, and a hybridization reaction unit 4. Further, on an upstream side of the nucleic acid amplification unit 3, there is provided a sample stage 7 allowing arrangement of nucleic acid sample standby wells 8 for temporarily placing the nucleic acid sample. A hybridization reaction unit 4 includes a hybridization stage 42 provided on a stage fixing member 41, a cassette holder 50 (see FIG. 2), a hybridization plate 43, and a temperature adjuster (not shown). The hybridization plate 43 has multiple wells 44, each well storing a reagent needed for hybridization reaction.

In the following, an operation of the device of the present invention will be described with reference as appropriate to the drawings. In this embodiment, description will be made of a case where hybridization reaction is effected after performing nucleic acid amplification processing twice. More specifically, in the example described below, the nucleic acid contained in the nucleic acid sample is amplified by using the PCR method, and then impurities other than the target nucleic acid are removed; and then, the target nucleic acid is amplified again by using the PCR method, and hybridization reaction is effected.

First, the nucleic acid sample standby wells 8 loaded with nucleic acid samples are arranged on the sample stage 7, and a nucleic acid amplification plate 33 to be used, a pipette tip case 35, the hybridization reaction plate 43, and reaction cassettes 45 are respectively set. In each reaction cassette 45, there is provided a reaction chamber, where there is installed a DNA microarray.

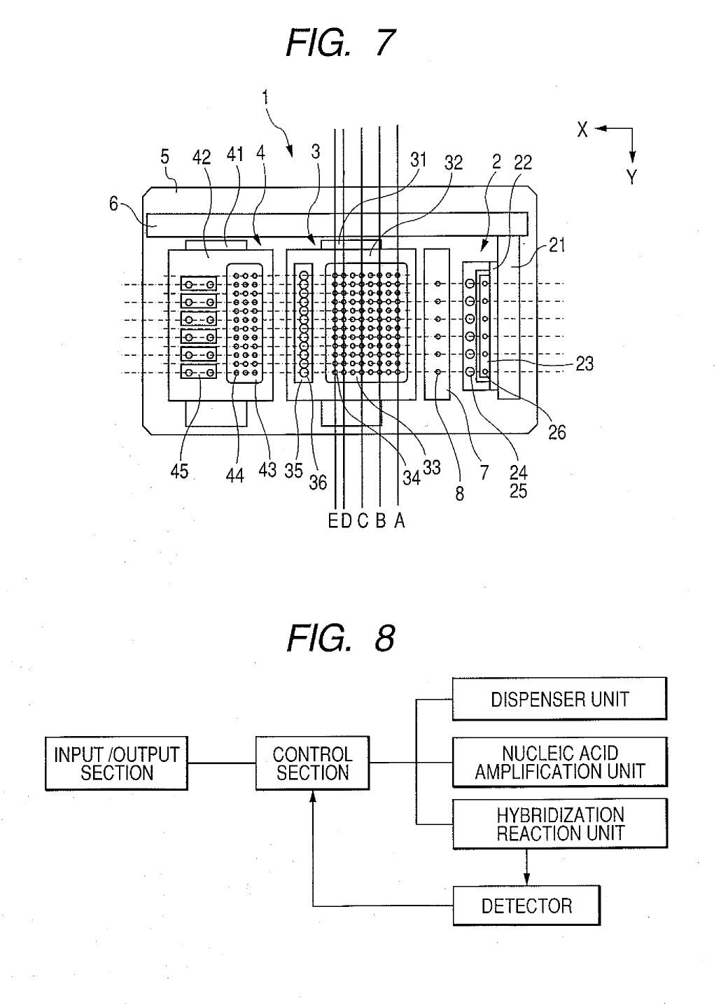

The dispenser unit 2 is supported by a dispenser unit guide 6 and a dispenser unit support member 21. The dispenser unit support member 21 is provided with a rail guide (not shown) extending in a Z-direction, enabling the dispenser unit 2 to move in the Z-direction. The dispenser unit 2 is capable of moving along a rail guide (not shown) in an X-direction through a space above the nucleic acid amplification unit 3 and the hybridization reaction unit 4. The dispenser unit 2 is formed by a casing 22 and a pipetting mechanism 24 generating negative pressure in the nozzles, and there are provided nozzle portions 25 allowing attachment of pipette chips to the forward ends of the pipetting mechanism 24. As shown in FIG. 1, the nozzle portions 25 are arranged at equal intervals in the Y-direction. FIG. 7 is a plan view showing the arrangement of each unit in the nucleic acid automatic examining device of this embodiment of the present invention.

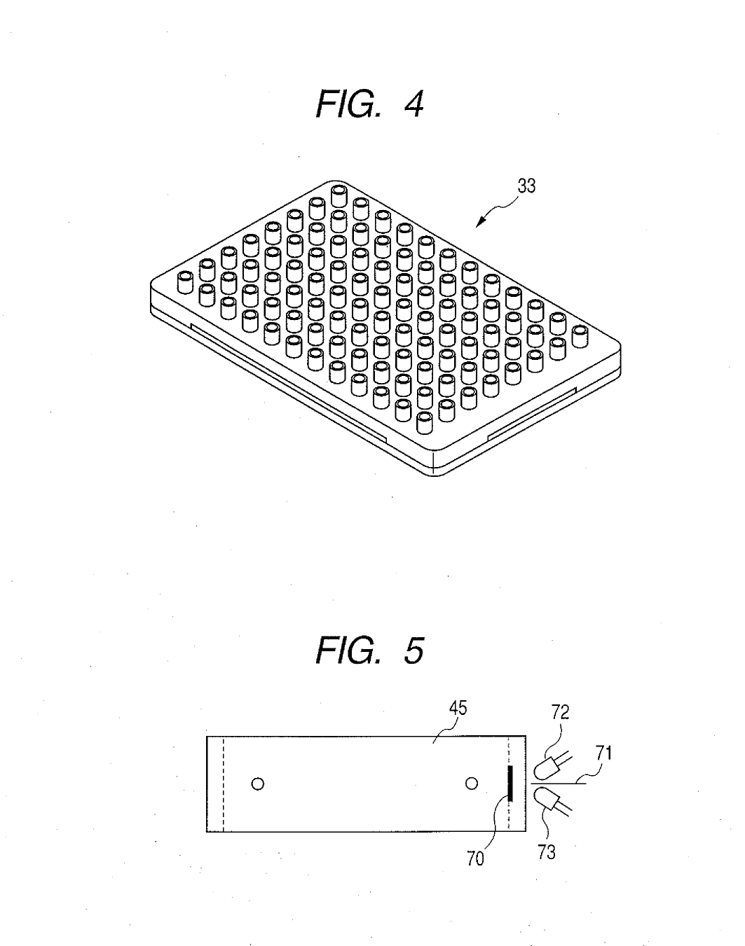

On a nucleic acid amplification stage 32, there can be arranged a nucleic acid amplification plate 33 having multiple amplification wells 34, and the pipette chip case 35. As the nucleic acid amplification plate 33, it is possible to employ, for example, a commercially available 96-well plate of polypropylene as shown in FIG. 4, which is equipped with 8×12 amplification wells 34. Here, as shown in FIG. 7, it is to be assumed that there are previously stored a 1st PCR reagent for use in 1st PCR in a line A, magnetic particles for use in the purification step in a line B, a cleaning liquid in a line C, an elution solution in a line D, and a 2nd PCR reagent for use in 2nd PCR in a line E.

The primer used when executing the PCR method and the magnetic particles and the elution solution used in the purification step may be well-known ones, a detailed description thereof with specific names mentioned will be omitted. Further, the arrangement of the reagents is naturally not restricted to that of the above-described example. To prevent intrusion of impurities, a protective sheet (not shown) may be attached to an upper side of the amplification wells 34. The pipette chip case 35 accommodates twelve pipette chips 36 in a row in a Y-direction. As indicated by dashed lines in FIG. 7, the pipette chips 36 are arranged in alignment with rows of the amplification wells 34.

First, the dispenser unit is moved by a drive unit (not shown) to attach the pipette chips 36 to the nozzle portions 25. The dispenser unit is moved such that the nozzle portions 25 and the pipette chips 36 are matched in their positions in the X-direction, and further, the dispenser unit 2 is lowered by the dispenser unit support member 21. As a result, the nozzle portions 25 and the pipette chips 36 are fit-engaged with each other.

The liquid dispensing operation in the present invention is conducted by using the dispenser unit. First, the dispenser unit 2 is driven along the rail guides (not shown) provided on the dispenser unit guide 6 and the dispenser unit support member 21 to bring the forward ends of the pipette chips attached to the nozzle portions 25 into contact with the liquid. The pipetting mechanism 24 is operated to generate negative pressure in the nozzles, thereby sucking the nucleic acid sample into the pipette chips. With the liquid being retained in the pipette chips, the dispenser unit support member 21 is driven again to discharge the retained liquid at desired positions.

By using the dispenser unit 2, the nucleic acid sample is moved from the nucleic acid sample standby wells 8 to the amplification wells 34 in the line A containing the 1st PCR reagent.

The nucleic acid amplification unit 3 includes a thermal cycler (not shown), and a nucleic acid amplification stage 32 capable of temperature adjustment by the thermal cycler. The nucleic acid amplification stage 32 is retained so as to be movable in the Y-direction with respect to a stationary member 31.

The dispenser unit is driven to dispense the nucleic acid sample from the nucleic acid sample standby wells 8 to the amplification wells in the line A. When the 1st PCR reagent and the nucleic acid sample are mixed together in the amplification wells in the line A, a temperature cycle is applied to the amplification wells 34 by using the thermal cycler.

After the completion of the 1st PCR, a purification step for removing impurities other than the target nucleic acid is started. Magnetic particles specifically adsorbing the target nucleic acid are contained in the amplification wells 34 in the line B. Subsequently, the purification step will be described. The nucleic acid having undergone the 1st PCR is moved by using the dispenser unit 2 to the amplification wells 34 in the line B containing magnetic particles, causing the magnetic particles to adsorb the target nucleic acid. The magnetic particles are fixed to the bottom surfaces of the amplification wells 34 by a magnetic force generating unit (not shown), and the remaining solution is removed by the dispenser unit 2 and disposed of through a waste liquid port (not shown). Further, the cleaning liquid stored in the amplification wells 34 in the line C is moved to the line B by the dispenser unit, and the magnetic particles are cleaned with the cleaning liquid. The magnetic particles are again fixed to the bottom surfaces of the amplification wells 34 by the magnetic force generating unit (not shown), and the used cleaning liquid is removed and disposed of.

At this stage, it is desirable to replace the pipette chips in order to prevent mixing of impurities, such as nucleic acids other than the target nucleic acid, into the purified target nucleic acid.

By using the dispenser unit with new pipette chips attached thereto, the elution solution stored in amplification wells in the line D is poured into the line B. Due to the elution solution, the target nucleic acid that has been adsorbed to the magnetic particles is liberated into the elution solution. The magnetic particles are fixed to the bottom surfaces of the amplification wells 34 by the magnetic force generating unit (not shown), and the liquid containing the liberated target nucleic acid is sucked by the dispenser unit 2, whereby the purification step is completed.

After the completion of the purification step, the sucked target nucleic acid is mixed with the 2nd PCR reagent contained in the amplification wells 34 in the line E, and a temperature cycle is applied thereto by the thermal cycler (not shown). This causes the 2nd PCR to proceed. In the 2nd PCR, exclusively the target nucleic acid is amplified. After the temperature cycle has been applied, the nucleic acid amplification step is all completed.

After the completion of the nucleic acid amplification step, the procedure advances to the hybridization step. The hybridization reaction unit 4 includes the stage fixing member 41 and the hybridization stage 42 mounted thereon. The hybridization stage 42 is movable in the Y-direction with respect to the stage fixing member 41. The hybridization plate 43 and the reaction cassette 45 can be arranged on the hybridization stage 42. As the hybridization plate 43, it is possible, for example, to employ one cut out from a commercially available 96-well plate of polypropylene as shown in FIG. 4 and equipped with 3×12 hybridization wells 44.

The nucleic acid solution having undergone the 2nd PCR is moved by the dispenser unit 2 to the hybridization wells 44 containing the hybridization reagent. When the nucleic acid solution is mixed with the hybridization reagent, the resultant mixture solution is transferred to the reaction cassettes 45. The reaction cassettes 45 are reaction cassettes which have reaction chambers where DNA microarrays are fixed, with hybridization reaction being effected within the reaction chambers. The reaction cassettes 45 are designed so that the target nucleic acid can be detected by utilizing the hybridization reaction. As the reaction cassettes 45 of this embodiment, it is possible to use well-known biochemical reaction cassettes, so a detailed description of the inner structure thereof will be omitted here.

As already stated above, when the reaction cassettes 45 are not set at predetermined positions in the cassette holders 50, futile standby time is generated when the processing in each step of the nucleic acid is stopped. To solve this problem, the detector first detects whether the reaction cassettes 45 are set in the cassette holder 50, and the dispenser unit and the nucleic acid amplification unit are controlled based on a detection signal output from the detector.

FIG. 3 is a schematic view illustrating the dispenser unit operating process and the nucleic acid sample examination process side by side. When the reaction cassette are not set at predetermined positions, the dispenser unit 2 is controlled so as to operate up to the nucleic acid amplification step but to stop at a stop line in transition to the hybridization step.

Actually, when the detector does not detect the reaction cassettes 45 set at predetermined positions in the cassette holder 50, the amplification unit and the dispenser unit operate up to the nucleic acid amplification step but do not advance to the hybridization step. The dispenser unit stops and an alarm indicator issues an alarm. The alarm indicator may be a lamp that is lighted, or one using voice. There are no particular limitations regarding the specific construction of the alarm indicator. With this construction, the user of the nucleic acid automatic examining device of the present invention can quickly ascertain stopping of the device. By accurately setting the reaction cassettes at predetermined positions, stopping of the dispenser unit 2 is detected by the detector, and control is effected so as to re-start the operation of the dispenser unit based on the detection signal therefrom. The control of the dispenser unit is conducted based on the detection signal issued upon detection of the reaction cassette 45 immediately before transition to the hybridization step.

FIG. 8 is a block diagram showing the relationship between the units related to the nucleic acid automatic examining device in this embodiment. After the positions of the reaction cassettes set in the cassette holder in the hybridization reaction unit are detected by the detector, the control section controls the dispenser unit, the nucleic acid amplification unit, and hybridization reaction unit based on the detection signal.

In the following, a method of detecting whether the reaction cassettes 45 are set at the accurate positions in the nucleic acid automatic examining device of this embodiment will be described.

FIG. 2 is a diagram showing the cassette holder 50 to which the reaction cassettes 45 are to be attached according to this embodiment of the present invention. In order that multiple cassettes may be aligned and set at the accurate positions, the reaction cassettes 45 are first attached to the cassette holder 50, and are then arranged on the hybridization stage 42. When hybridization reaction is completed and the reaction cassettes 45 are moved to the detection unit, they are moved while attached to the cassette holder 50.

To effect hybridization reaction, it is necessary for the reaction cassettes 45 to be held in intimate contact with a temperature adjuster. For that purpose, it is desirable for the reaction cassettes 45 to be individually separated from each other so that they allow equalization in conformity with the surface of the temperature adjuster with a certain degree of freedom within the cassette holder 50. Further, when optically detecting the hybrid of the target nucleic acid by the detection unit, it is necessary to perform focusing and tilt adjustment.

FIG. 5 is a diagram showing an example of the sensor for detecting the cassette. The sensor is an optical detection sensor for detecting whether the reaction cassette 45 is set by using a light emitting element 72, a light receiving element 73, and a reflection plate 70. In this optical detection sensor, light emitted from the light emitting element 72 is reflected by the reflection plate 70 mounted to the reaction cassette 45, and the reflection light is received by the light receiving element, whereby the cassette is detected. Therefore, the light emitting element 72, the light receiving element 73, and the reflection plate 70 are arranged such that the above-mentioned optical path is formed only when the reaction cassette 45 is set at a predetermined attachment position on the hybridization reaction unit or the detection unit. Further, it is also possible to provide a shielding plate 71 for cutting off light from the light emitting element transmitted without being reflected by the reflection plate.

As the light emitting element 72, it is possible to use, for example, a light emitting diode. As the light receiving element 73, it is possible to use, for example, a photo transistor. There are no particular limitations regarding the reflection plate 70 as long as it is formed of a material capable of reflecting light emitted from the light emitting element.

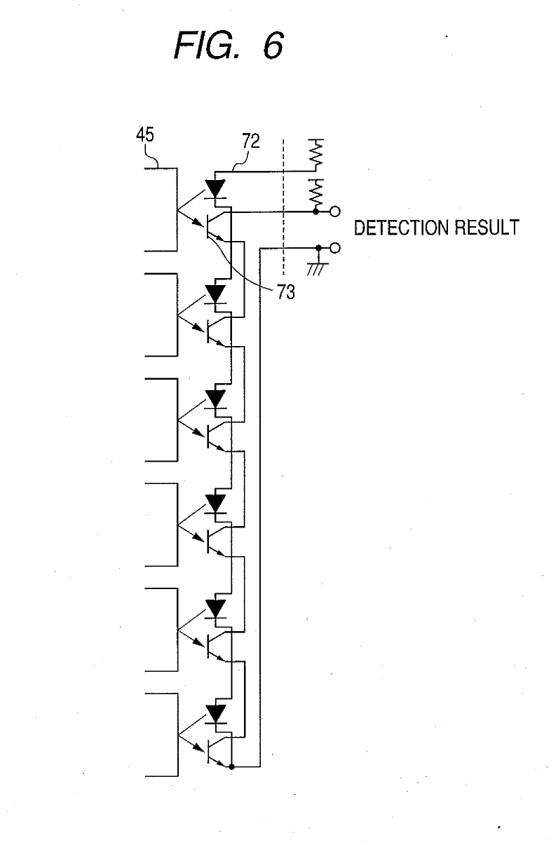

FIG. 6 is a diagram showing an example of the detection circuit including the above-mentioned detection sensor. This detection circuit is an electric circuit in which the above-mentioned detection sensor is installed for each of multiple cassettes to be detected, with the detection sensors being connected as shown in the drawing. In the case of the circuit of FIG. 6, when all the cassettes are attached, the voltage level of the circuit as the detection result is L.

If even a single cassette is left unattached, the voltage level of the circuit as the detection result is H. To actually install the detection circuit of FIG. 6, it is possible, for example, to previously incorporate the light emitting elements 72 and the light receiving elements 73 in the cassette holder 50. When the cassette holder 50 is arranged on the hybridization stage 42, electric contacts provided on both of them come into contact with each other to effect exchange of signals to thereby detect the reaction cassettes 45. Alternatively, it is also possible to arrange the light emitting elements 72 and the light receiving elements 73 at positions on the hybridization stage 42 in the vicinity of the place where the cassette holder 50 is to be arranged, detecting the reaction cassettes 45 when the cassette holder 50 is arranged. The point of time when the detector operates may, for example, be when the cassette holder 50 with the reaction cassettes 45 attached thereto is set on the hybridization stage 42, and the cover is closed, or when a setting completion button or an examination start button is depressed. In the case in which the detection result is H, it is also possible to indicate alarm without starting the examination. For example, it may be lighting-up or flashing of a lamp, lighting-up or flashing of a sign on a display screen such as an LCD, or a display of a warning. Further, in addition to those, it is also possible to use an alarm sound such as that of a buzzer.

While in the example of FIG. 6 the light emitting elements and the light receiving elements are connected in series to thereby detect whether all the reaction cassettes have been arranged at predetermined positions, it is also possible to connect sensors each formed of a combination of a pair of light emitting element and light receiving element in parallel, effecting detection at each individual position. Alternatively, it is also possible to adopt a construction in which one or several sensors are moved relative to the reaction cassette installation positions to detect the arrangement condition of the reaction cassettes.

The method of detecting the reaction cassettes 45 is not restricted to the optical methods as described above. For example, it would be also possible to detect the attachment of the reaction cassettes 45 to the cassette holder 50 by an electrical detection sensor through conduction when electric contacts provided on both of them come into contact with each other, or by a mechanical switch.

In the nucleic acid automatic examining device of this embodiment, the hybridization stage 42 is mounted on the stage fixing member 41 and is movable in the Y-direction. In the case of cassette detection utilizing this construction, a set of light emitting element 72 and light receiving element 73 are arranged on the main body of the examining device. In this case, after moving the hybridization stage 42 to the front side to set the cassette holder 50, it is possible to detect the reaction cassettes 45 one by one while moving to the detecting position, thus detecting whether all the reaction cassettes 45 have been attached.

In the nucleic acid automatic examining device according to the present invention, it is possible to automate the examination through computer control of each operation according to a pre-set program. The specific setting of each operation may be effected as appropriate through programming according to the kind of target nucleic acid to be examined, the construction of the detection system, etc.

While in the above embodiment the present invention is applied, by way of example, to a nucleic acid automatic examining device which has reaction steps such as nucleic acid amplification through PCR and hybridization reaction, and which uses a DNA microarray, this should not be construed restrictively.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-130297, filed May 9, 2006, which is hereby incorporated by reference herein in its entirety.

What is claimed is:

1. A nucleic acid automatic examining device for implementing a hybridization reaction step after implementing a nucleic acid amplification reaction step on a nucleic acid sample containing a target nucleic acid, comprising:
a nucleic acid amplification unit for implementing the nucleic acid amplification reaction step;
a hybridization reaction unit having a cassette holder allowing installation of a reaction cassette for use in the hybridization reaction step performed on the nucleic acid sample;
a movable dispenser unit having multiple nozzles capable of sucking and discharging the nucleic acid sample and moving the nucleic acid sample from the nucleic acid amplification unit to the hybridization reaction unit;
a detector for detecting presence or absence of the reaction cassette in the cassette holder; and
an alarm indicator for indicating an alarm,
wherein the nucleic acid amplification unit and the hybridization reaction unit are operable in parallel and are controlled such that, when the detector detects the absence of the reaction cassette, the device implements steps up to the nucleic acid amplification reaction step and does not implement the subsequent hybridization reaction step while the alarm indicator indicates an alarm and the dispenser unit is stopped.

2. A nucleic acid automatic examining device according to claim 1, wherein the device is capable of further implementing a nucleic acid purification step after the nucleic acid amplification reaction step and before the hybridization reaction step.

3. A nucleic acid automatic examining device according to claim 1, wherein the device is controlled such that, when the reaction cassette is detected after the dispenser unit is stopped, the dispenser unit re-starts moving the nucleic acid sample from the nucleic acid amplification unit to the hybridization reaction unit based on a detection signal.

4. A nucleic acid automatic examining device for implementing a hybridization reaction step after implementing a nucleic acid amplification reaction step on a nucleic acid sample containing a target nucleic acid, comprising:
a nucleic acid amplification unit for implementing the nucleic acid amplification reaction step;
a hybridization reaction unit having a cassette holder allowing installation of a reaction cassette for use in the hybridization reaction step performed on the nucleic acid sample;
a movable dispenser unit having multiple nozzles capable of sucking and discharging the nucleic acid sample and moving the nucleic acid sample from the nucleic acid amplification unit to the hybridization reaction unit;
a detector for detecting presence or absence of the reaction cassette in the cassette holder; and
an alarm indicator for indicating an alarm,
wherein the device is controlled such that, when the detector detects the absence of the reaction cassette, the device implements steps up to the nucleic acid amplification reaction step and does not implement the subsequent hybridization reaction step while the alarm indicator indicates an alarm and the dispenser unit is stopped.

5. A nucleic acid automatic examining device for implementing a hybridization reaction step after implementing a nucleic acid amplification reaction step on a nucleic acid sample containing a target nucleic acid, comprising:
a nucleic acid amplification unit for implementing the nucleic acid amplification reaction step;
a hybridization reaction unit having a cassette holder allowing installation of a reaction cassette for use in the hybridization reaction step performed on the nucleic acid sample;
a movable dispenser unit having multiple nozzles capable of sucking and discharging the nucleic acid sample and moving the nucleic acid sample from the nucleic acid amplification unit to the hybridization reaction unit; and
a detector for detecting presence or absence of the reaction cassette in the cassette holder,
wherein the nucleic acid amplification unit and the hybridization reaction unit are operable in parallel and are controlled such that, when the detector detects the absence of the reaction cassette, the device implements steps up to the nucleic acid amplification reaction step and does not implement the subsequent hybridization reaction step while the dispenser unit is stopped.

6. A nucleic acid automatic examining device for implementing a hybridization reaction step after implementing a nucleic acid amplification reaction step on a nucleic acid sample containing a target nucleic acid, comprising:
a nucleic acid amplification unit for implementing the nucleic acid amplification reaction step;
a hybridization reaction unit having a cassette holder allowing installation of a reaction cassette for use in the hybridization reaction step performed on the nucleic acid sample;
a movable dispenser unit having multiple nozzles capable of sucking and discharging the nucleic acid sample and moving the nucleic acid sample from the nucleic acid amplification unit to the hybridization reaction unit; and
a detector for detecting presence or absence of the reaction cassette in the cassette holder,
wherein the device is controlled such that, when the detector detects the absence of the reaction cassette, the device implements steps up to the nucleic acid amplification reaction step and does not implement the subsequent hybridization reaction step while the dispenser unit is stopped.
